(19) United States
(12) Patent Application Publication
(10) Pub. No.: US 2020/0113544 A1
(43) Pub. Date: Apr. 16, 2020

(54) METHOD AND SYSTEM FOR ENHANCED VISUALIZATION OF ULTRASOUND PROBE POSITIONING FEEDBACK

(71) Applicant: General Electric Company, Schenectady, NY (US)

(72) Inventors: Thomas Huepf, Zipf (AT); Johann Himsl, Zipf (AT); Stefan Denk, Zipf (AT)

(21) Appl. No.: 16/160,316

(22) Filed: Oct. 15, 2018

(51) Int. Cl. A61B 8/00; A61B 8/08; G06T 7/00; G06T 7/60; G06T 7/70

(52) U.S. Cl. CPC A61B 8/463; A61B 8/4254; A61B 8/5215; G06T 7/0012; G06T 7/60; G06T 2207/30004; A61B 8/08; G06T 2207/10132; G06T 2207/20081; G06T 2207/20221; G06T 7/70

(57) ABSTRACT

A system and method for providing enhanced visualization of ultrasound probe positioning feedback is provided. The method includes displaying a mask defining a target position and orientation of an ultrasound probe that corresponds to a pre-defined view of anatomical structure. The mask includes a primary target area, lateral target area(s) positioned laterally from the primary target area, and elevational target area(s) positioned in an elevational direction from the primary target area. The method includes displaying a reticle having a reticle position and orientation corresponding to a position and orientation of the probe. The reticle position and orientation is dynamically updated with respect to the mask based on the probe position data and in response to movement of the probe. The reticle includes a primary reticle element, lateral reticle element(s) positioned laterally from the primary reticle element, and elevational reticle element(s) positioned in an elevational direction from the primary reticle element.

### FIELD

Certain embodiments relate to ultrasound imaging. More specifically, certain embodiments relate to a method and system for providing visual feedback related to the positioning of an ultrasound probe to obtain desired ultrasound image views. The visual feedback may include a mask corresponding to a target position and orientation for the ultrasound probe and a reticle corresponding to a current position and orientation of the ultrasound probe. The mask and reticle may be superimposed on ultrasound data with the reticle position and orientation dynamically updating in response to movement of the ultrasound probe. The ultrasound operator may move the ultrasound probe based on the feedback until the reticle is aligned with the mask.

### BACKGROUND

Ultrasound imaging is a medical imaging technique for imaging organs and soft tissues in a human body. Ultrasound imaging uses real time, non-invasive high frequency sound waves to produce a two-dimensional (2D) image and/or a three-dimensional (3D) image.

During an ultrasound imaging examination, an ultrasound operator may manipulate an ultrasound probe to scan an ultrasound volume-of-interest from different positions and orientations. For example, an ultrasound operator may manipulate a probe to acquire images of a fetal heart from multiple different positions and orientations. However, correctly orienting the probe in order to acquire images of the desired volume-of-interest from the different positions may be challenging, particularly for inexperienced operators. The anatomical structures of a patient may appear different from various perspectives and there are several degrees of freedom (position, rotation, and tilt) for adjusting the probe. The difficulty in locating and scanning the desired volume-of-interest from different probe positions may result in a longer total scan time to complete an ultrasound examination, even for an experienced user.

Further limitations and disadvantages of conventional and traditional approaches will become apparent to one of skill in the art, through comparison of such systems with some aspects of the present disclosure as set forth in the remainder of the present application with reference to the drawings.

### BRIEF SUMMARY

A system and/or method is disclosed for providing enhanced visualization of ultrasound probe positioning feedback, substantially as shown in and/or described in connection with at least one of the figures, as set forth more completely in the claims.

These and other advantages, aspects and novel features of the present disclosure, as well as details of an illustrated embodiment thereof, will be more fully understood from the following description and drawings.

### BRIEF DESCRIPTION OF SEVERAL VIEWS OF THE DRAWINGS

FIG. 1 is a block diagram of an exemplary ultrasound system that is operable to provide enhanced visualization of ultrasound probe positioning feedback, in accordance with various embodiments.

FIG. 2 illustrates an exemplary mask and reticle configured to provide enhanced visualization of ultrasound probe positioning feedback, in accordance with exemplary embodiments.

FIG. 3 illustrates an exemplary reticle aligned with an exemplary mask that corresponds with a correctly positioned ultrasound probe, in accordance with various embodiments.

FIG. 4 illustrates an exemplary reticle laterally misaligned with an exemplary mask to provide feedback for moving an ultrasound probe to a correct position and orientation, in accordance with exemplary embodiments.

FIG. 5 illustrates an exemplary reticle misaligned in an elevation direction with an exemplary mask to provide feedback for moving an ultrasound probe to a correct position and orientation, in accordance with various embodiments.

FIG. 6 illustrates an exemplary reticle rotationally misaligned with an exemplary mask to provide feedback for moving an ultrasound probe to a correct position and orientation, in accordance with exemplary embodiments.

FIG. 7 illustrates an exemplary reticle having a lateral tilt, in accordance with various embodiments.

FIG. 8 illustrates an exemplary reticle having an elevational tilt, in accordance with exemplary embodiments.

FIG. 9 illustrates exemplary masks having different precision levels, in accordance with various embodiments.

FIG. 10 illustrates an exemplary mask and reticle overlaid on an ultrasound image to provide enhanced visualization of ultrasound probe positioning feedback, in accordance with exemplary embodiments.

FIG. 11 is a flow chart illustrating exemplary steps that may be utilized for providing enhanced visualization of ultrasound probe positioning feedback, in accordance with various embodiments.

### DETAILED DESCRIPTION

Certain embodiments may be found in a method and system for positioning an ultrasound probe. Various embodiments have the technical effect of providing visual feedback for positioning a probe to capture desired ultrasound image data. Moreover, certain embodiments have the technical effect of converting a position and orientation of an ultrasound probe to a single reticle for alignment with a single mask. The single mask may provide target areas defining the appropriate position, rotation, tilt, and an amount of precision associated with each of these elements. The single reticle may provide elements to present visual feedback with respect to the current position, rotation, and tilt of the ultrasound probe. Furthermore, various embodiments have the technical effect of automating an imaging system action once an ultrasound probe is detected in a correct position and orientation for obtaining desired ultrasound image data. For example, once a reticle corresponding with a position and orientation of an ultrasound probe is aligned with a mask corresponding with a desired view of a volume of interest, the ultrasound system may be configured to automatically store the acquired ultrasound image data, automatically provide tools for performing a measurement, and/or automatically perform a measurement of anatomical structure in the acquired ultrasound image data, among other things.

The foregoing summary, as well as the following detailed description of certain embodiments will be better understood when read in conjunction with the appended drawings. To the extent that the figures illustrate diagrams of the functional blocks of various embodiments, the functional blocks are not necessarily indicative of the division between hardware circuitry. Thus, for example, one or more of the functional blocks (e.g., processors or memories) may be implemented in a single piece of hardware (e.g., a general purpose signal processor or a block of random access memory, hard disk, or the like) or multiple pieces of hardware. Similarly, the programs may be stand alone programs, may be incorporated as subroutines in an operating system, may be functions in an installed software package, and the like. It should be understood that the various embodiments are not limited to the arrangements and instrumentality shown in the drawings. It should also be understood that the embodiments may be combined, or that other embodiments may be utilized and that structural, logical and electrical changes may be made without departing from the scope of the various embodiments. The following detailed description is, therefore, not to be taken in a limiting sense, and the scope of the present disclosure is defined by the appended claims and their equivalents.

As used herein, an element or step recited in the singular and proceeded with the word “a” or “an” should be understood as not excluding plural of said elements or steps, unless such exclusion is explicitly stated. Furthermore, references to “an exemplary embodiment,” “various embodiments,” “certain embodiments,” “a representative embodiment,” “one embodiment,” and the like, are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional elements not having that property.

Also as used herein, the term “image” broadly refers to both viewable images and data representing a viewable image. However, many embodiments generate (or are configured to generate) at least one viewable image. In addition, as used herein, the phrase “image” is used to refer to an ultrasound mode such as three-dimensional (3D) mode, B-mode, CF-mode, and/or sub-modes of B-mode and/or CF such as Shear Wave Elasticity Imaging (SWEI), TVI, Angio, B-flow, BMI, BMI_Angio, and in some cases also MM, CM, PW, TVD, CW where the “image” and/or “plane” includes a single beam or multiple beams.

Furthermore, the term processor or processing unit, as used herein, refers to any type of processing unit that can carry out the required calculations needed for the various embodiments, such as single or multi-core: CPU, Graphics Board, DSP, FPGA, ASIC or a combination thereof.

It should be noted that various embodiments described herein that generate or form images may include processing for forming images that in some embodiments includes beamforming and in other embodiments does not include beamforming. For example, an image can be formed without beamforming, such as by multiplying the matrix of demodulated data by a matrix of coefficients so that the product is the image, and wherein the process does not form any “beams”. Also, forming of images may be performed using channel combinations that may originate from more than one transmit event (e.g., synthetic aperture techniques).

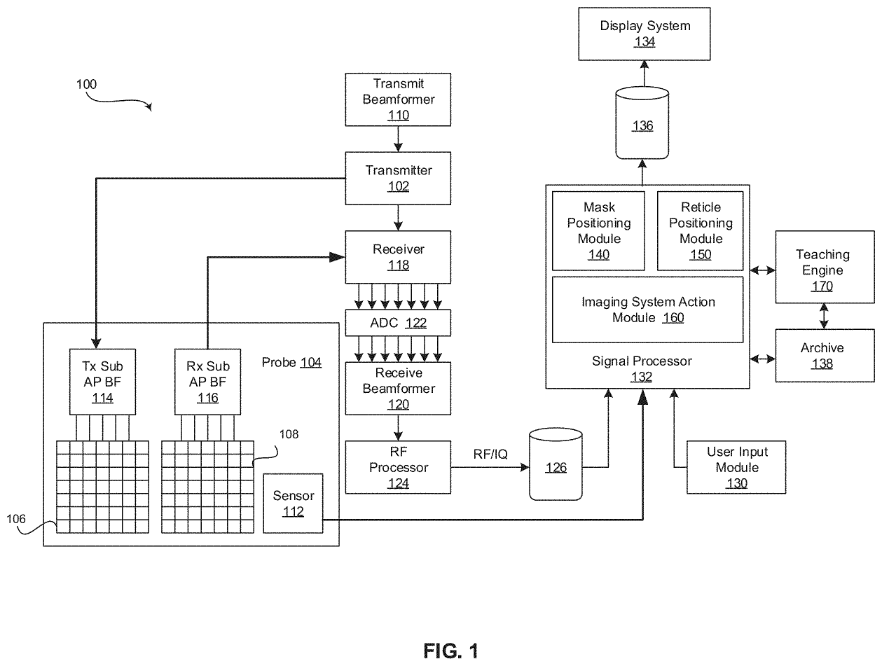

In various embodiments, ultrasound processing to form images is performed, for example, including ultrasound beamforming, such as receive beamforming, in software, firmware, hardware, or a combination thereof. One implementation of an ultrasound system having a software beamformer architecture formed in accordance with various embodiments is illustrated in FIG. 1.

FIG. 1 is a block diagram of an exemplary ultrasound system 100 that is operable to provide enhanced visualization of ultrasound probe 104 positioning feedback, in accordance with various embodiments. Referring to FIG. 1, there is shown an ultrasound system 100. The ultrasound system 100 comprises a transmitter 102, an ultrasound probe 104, a position sensing system 112, a transmit beamformer 110, a receiver 118, a receive beamformer 120, a RF processor 124, a RF/IQ buffer 126, a user input module 130, a signal processor 132, an image buffer 136, a display system 134, an archive 138, and a teaching engine 170.

The transmitter 102 may comprise suitable logic, circuitry, interfaces and/or code that may be operable to drive an ultrasound probe 104. The ultrasound probe 104 may comprise a two dimensional (2D) array of piezoelectric elements. The ultrasound probe 104 may comprise a group of transmit transducer elements 106 and a group of receive transducer elements 108, that normally constitute the same elements. The ultrasound system 100 may include a position sensing system 112 attached to the probe 104. The position sensing system 112 may include an optical tracking system, magnetic position system, a sensor in a probe holder, a motion sensing system, and/or any suitable system or combinations of systems configured to detect the position and orientation of the probe 104. For example, the ultrasound system 100 may include an external magnetic field generator comprising a coil and/or a permanent magnet that when energized, may generate a static external magnetic field. The position sensing system 112 may be configured to detect a preexisting magnetic field or the magnetic field generated by the external magnetic field generator. The external magnetic field generator may be configured to generate a magnetic field with a gradient so that the position of the magnetic position sensor may be determined based on the detected magnetic field. In various embodiments, the position sensing system 112 may provide the probe position data to the signal processor 132 of the ultrasound system 100 for association with ultrasound image data acquired by the ultrasound probe 104 at the corresponding probe positions and orientations and/or to generate a reticle 300 corresponding with the probe position and orientations as discussed in more detail below. In certain embodiment, the ultrasound probe 104 may be operable to acquire ultrasound image data covering anatomical structure, such as a fetus, a fetal heart, a liver, a heart, or any suitable organ or other anatomical structure.

The transmit beamformer 110 may comprise suitable logic, circuitry, interfaces and/or code that may be operable to control the transmitter 102 which, through a transmit sub-aperture beamformer 114, drives the group of transmit transducer elements 106 to emit ultrasonic transmit signals into a region of interest (e.g., human, animal, underground cavity, physical structure and the like). The transmitted ultrasonic signals may be back-scattered from structures in the object of interest, like blood cells or tissue, to produce echoes. The echoes are received by the receive transducer elements 108.

The group of receive transducer elements 108 in the ultrasound probe 104 may be operable to convert the received echoes into analog signals, undergo sub-aperture beamforming by a receive sub-aperture beamformer 116 and are then communicated to a receiver 118. The receiver 118 may comprise suitable logic, circuitry, interfaces and/or code that may be operable to receive and demodulate the signals from the receive sub-aperture beamformer 116. The demodulated analog signals may be communicated to one or more of the plurality of A/D converters 122.

The plurality of A/D converters 122 may comprise suitable logic, circuitry, interfaces and/or code that may be operable to convert the demodulated analog signals from the receiver 118 to corresponding digital signals. The plurality of A/D converters 122 are disposed between the receiver 118 and the receive beamformer 120. Notwithstanding, the disclosure is not limited in this regard. Accordingly, in some embodiments, the plurality of A/D converters 122 may be integrated within the receiver 118.

The receive beamformer 120 may comprise suitable logic, circuitry, interfaces and/or code that may be operable to perform digital beamforming processing to, for example, sum the delayed channel signals received from the plurality of A/D converters 122 and output a beam summed signal. The resulting processed information may be converted back to corresponding RF signals. The corresponding output RF signals that are output from the receive beamformer 120 may be communicated to the RF processor 124. In accordance with some embodiments, the receiver 118, the plurality of A/D converters 122, and the beamformer 120 may be integrated into a single beamformer, which may be digital.

The RF processor 124 may comprise suitable logic, circuitry, interfaces and/or code that may be operable to demodulate the RF signals. In accordance with an embodiment, the RF processor 124 may comprise a complex demodulator (not shown) that is operable to demodulate the RF signals to form I/Q data pairs that are representative of the corresponding echo signals. The RF or I/Q signal data may then be communicated to an RF/IQ buffer 126. The RF/IQ buffer 126 may comprise suitable logic, circuitry, interfaces and/or code that may be operable to provide temporary storage of the RF or I/Q signal data, which is generated by the RF processor 124.

The user input module 130 may be utilized to patient data, scan parameters, settings, select protocols and/or templates, identify anatomical structure in ultrasound image data, perform measurements, and the like. In an exemplary embodiment, the user input module 130 may be operable to configure, manage and/or control operation of one or more components and/or modules in the ultrasound system 100. In this regard, the user input module 130 may be operable to configure, manage and/or control operation of the transmitter 102, the ultrasound probe 104, the transmit beamformer 110, the position sensing system 112, the receiver 118, the receive beamformer 120, the RF processor 124, the RF/IQ buffer 126, the user input module 130, the signal processor 132, the image buffer 136, the display system 134, the archive 138, and/or the teaching engine 170. The user input module 130 may include button(s), a touchscreen, motion tracking, voice recognition, a mousing device, keyboard, camera and/or any other device capable of receiving a user directive. In certain embodiments, one or more of the user input modules 130 may be integrated into other components, such as the display system 134, for example. As an example, user input module 130 may include a touchscreen display. In various embodiments, anatomical structure in ultrasound image data may be selected in response to a directive received via the user input module 130. In certain embodiments, measurements of anatomical structure in ultrasound data may be performed in response to a directive received via the user input module 130 to, for example, select a particular measurement, select caliper start and end point positions, and/or define a measurement area in the ultrasound image data.

The signal processor 132 may comprise suitable logic, circuitry, interfaces and/or code that may be operable to process ultrasound scan data (i.e., RF signal data or IQ data pairs) for generating ultrasound images for presentation on a display system 134. The signal processor 132 is operable to perform one or more processing operations according to a plurality of selectable ultrasound modalities on the acquired ultrasound scan data. In an exemplary embodiment, the signal processor 132 may be operable to perform compounding, motion tracking, and/or speckle tracking. Acquired ultrasound scan data may be processed in real-time during a scanning session as the echo signals are received. Additionally or alternatively, the ultrasound scan data may be stored temporarily in the RF/IQ buffer 126 during a scanning session and processed in less than real-time in a live or off-line operation. In various embodiments, each of the ultrasound images generated by the signal processor 132 may be associated with probe position data received from the probe position sensing system 112 of the ultrasound probe 104 to associate each of the ultrasound images with the position and orientation of the probe at the time of ultrasound image data acquisition. The processed image data and associated probe position data can be presented at the display system 134 and/or may be stored at the archive 138. The archive 138 may be a local archive, a Picture Archiving and Communication System (PACS), or any suitable device for storing images and related information. In the exemplary embodiment, the signal processor 132 may comprise a mask positioning module 140, a reticle positioning module 150, and an imaging system action module 160.

The ultrasound system 100 may be operable to continuously acquire ultrasound scan data at a frame rate that is suitable for the imaging situation in question. Typical frame rates range from 20-70 but may be lower or higher. The acquired ultrasound scan data may be displayed on the display system 134 at a display-rate that can be the same as the frame rate, or slower or faster. An image buffer 136 is included for storing processed frames of acquired ultrasound scan data that are not scheduled to be displayed immediately. Preferably, the image buffer 136 is of sufficient capacity to store at least several minutes' worth of frames of ultrasound scan data. The frames of ultrasound scan data are stored in a manner to facilitate retrieval thereof according to its order or time of acquisition. The image buffer 136 may be embodied as any known data storage medium.

The signal processor 132 may include a mask positioning module 140 that comprises suitable logic, circuitry, interfaces and/or code that may be operable to receive identification of and/or automatically identify anatomical structure in acquired ultrasound image data. For example, a user may manually identify anatomical structure in acquired ultrasound image data by providing a directive via the user input module 130. The user input module 130 may receive, for example, a user directive to select or otherwise identify a head, abdomen, or femur, among other things, in ultrasound image data of a fetus.

As another example, the mask positioning module 140 may include image detection algorithms, one or more deep neural networks and/or may utilize any suitable form of image detection techniques or machine learning processing functionality configured to automatically identify anatomical structure in the ultrasound image data. For example, the mask positioning module 140 may be made up of an input layer, an output layer, and one or more hidden layers in between the input and output layers. Each of the layers may be made up of a plurality of processing nodes that may be referred to as neurons. For example, the input layer may have a neuron for each pixel or a group of pixels from the ultrasound images of the anatomical structure. The output layer may have a neuron corresponding to each structure of the fetus or organ being imaged. As an example, if imaging a fetus, the output layer may include neurons for a head, abdomen, femur, unknown, and/or other. Each neuron of each layer may perform a processing function and pass the processed ultrasound image information to one of a plurality of neurons of a downstream layer for further processing. As an example, neurons of a first layer may learn to recognize edges of structure in the ultrasound image data. The neurons of a second layer may learn to recognize shapes based on the detected edges from the first layer. The neurons of a third layer may learn positions of the recognized shapes relative to landmarks in the ultrasound image data. The processing performed by the mask positioning module 140 deep neural network may identify anatomical structure in ultrasound image data with a high degree of probability.

In various embodiments, the mask positioning module 140 may comprise suitable logic, circuitry, interfaces and/or code that may be operable to generate and superimpose a mask on acquired ultrasound image data based on the identification of the anatomical structure. The mask may correspond with a pre-defined view of the particular anatomical structure. For example, the pre-defined view of a fetal head may be a cross-sectional view of the head at a level of the thalami with symmetrical appearance of both hemispheres and no cerebellum visualized. The view may have an angle of insonation of ninety (90) degrees to the midline echoes. The pre-defined view of the fetal head may provide a desired view to, for example, perform a biparietal diameter (BPD) measurement and/or a head circumference (HC) measurement. As another example, a pre-defined view of a fetal abdomen may be a transverse section of the fetal abdomen (as circular as possible) with the umbilical vein at a level of the portal sinus, the stomach bubble visualized, and kidneys not visible. The information regarding the pre-defined views of each anatomical structure may be stored in archive 138 or any suitable data storage medium. The mask positioning module 140 may access the information related to the pre-defined view of the identified anatomical structure and may generate and superimpose the mask corresponding to the pre-defined view on acquired ultrasound image data.

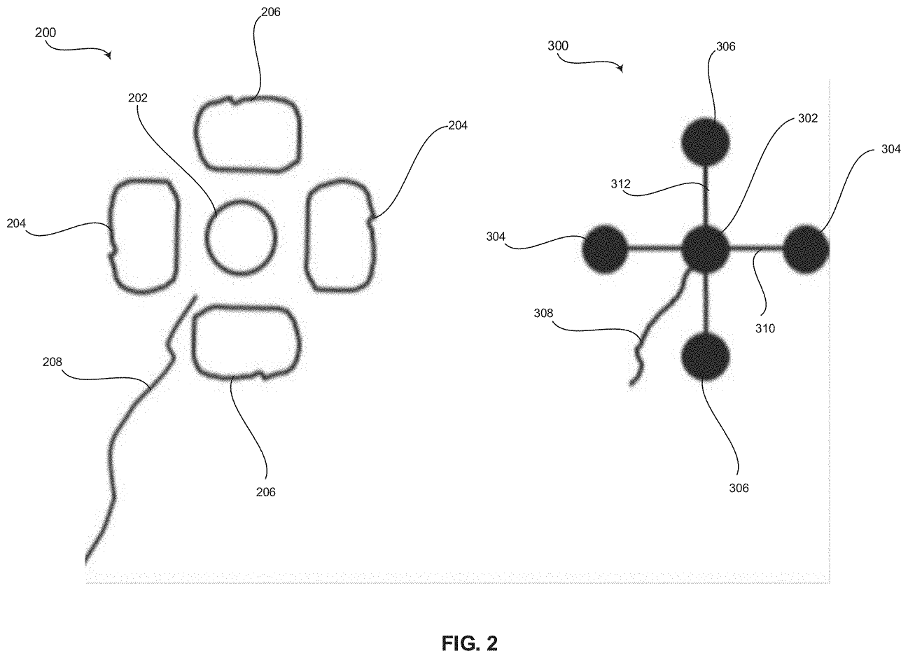

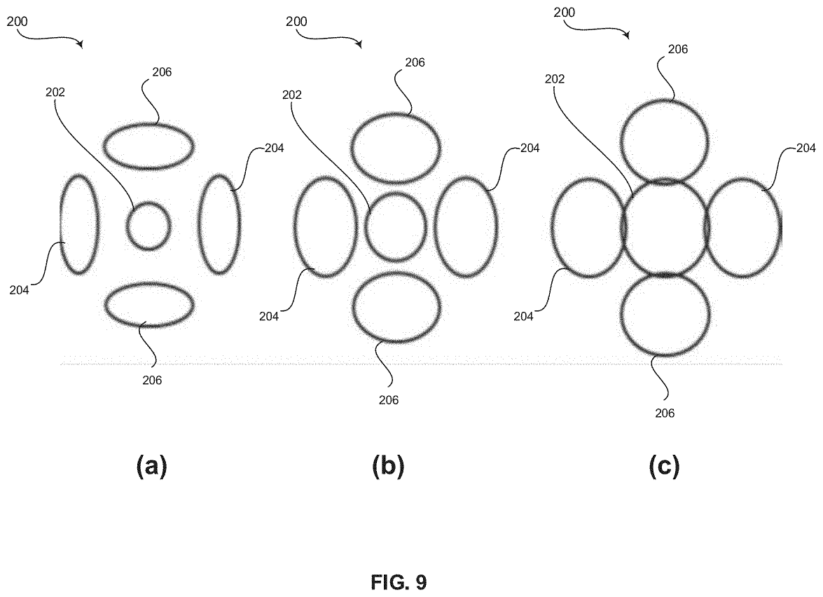

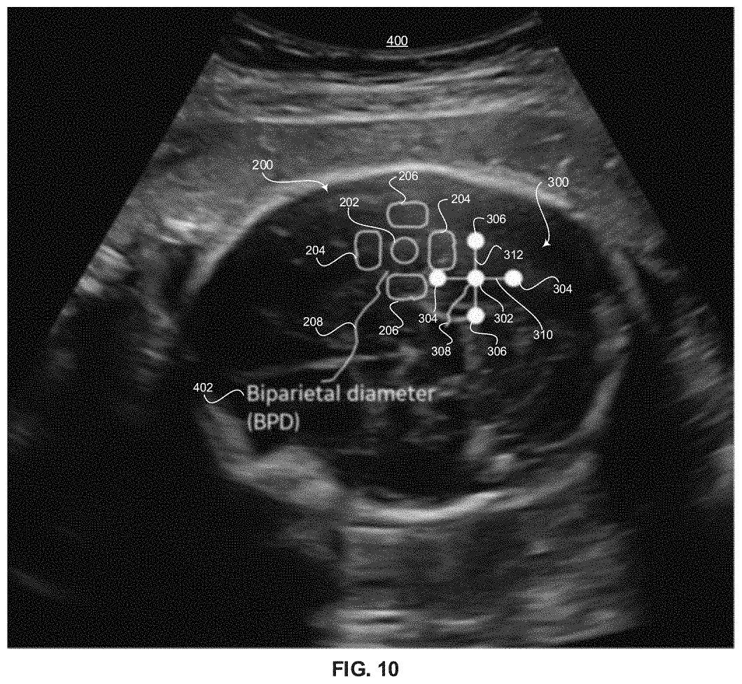

FIG. 9 illustrates exemplary masks 200 having different precision levels, in accordance with various embodiments. Referring to FIG. 9, each mask 200 may include a primary target area 202, at least one lateral target area 204, and at least one elevational target area 206. Each of the target areas 202, 204, 206 may be an enclosed shape, such as a circle, oval, square, rectangle, or any suitable shape. The at least one lateral target area 204 may be located on a first side, a second side, or both sides of the primary target area 202. The at least one elevational target area 206 may be located above the primary target area 202, below the primary target area 202, or both above and below the primary target area 202. In the exemplary embodiment illustrated in FIG. 9, the mask includes a centrally-located primary target area 202 with a lateral target area 204 on both sides of the primary target area 202 and an elevational target area 206 both above and below the primary target area 202. The alignment of the target areas 202, 204, 206 and a position of a mask rotational indicator 208 (as shown in FIGS. 2-6 and 10) corresponds with a target orientation (e.g., rotation and tilt) of an ultrasound probe 104 for obtaining the pre-defined view of the anatomical structure. The position of the mask 200 with respect to a position of a reticle 300 (shown in FIGS. 2-8 and 10) associated with the current position of the ultrasound probe 104 corresponds with a target position of the ultrasound probe 104 for obtaining the pre-defined view of the anatomical structure. The size of the target areas 202, 204, 206 corresponds with the amount of alignment precision for obtaining the pre-defined view of the anatomical structure. For example, smaller target areas may correspond with a higher level of alignment precision to obtain the pre-defined view than larger target areas. As described in more detail below, manipulating an ultrasound probe 104 to align the reticle 300 within the target areas 202, 204, 206 of the mask 200 results in the ultrasound probe 104 being positioned and oriented to obtain the desired pre-defined view of the anatomical structure. The mask 200 may be overlaid onto ultrasound image data 400 presented at the display system 134 as shown in FIG. 10 and described in more detail below. Additionally and/or alternatively, the mask 200 may be presented at other portions of an ultrasound display at the display system 134, such as in a side, top, or bottom panel of the display.

Referring again to FIG. 1, the signal processor 132 may include a reticle positioning module 150 that comprises suitable logic, circuitry, interfaces and/or code that may be operable to generate and superimpose a reticle 300 corresponding to a current position and orientation of an ultrasound probe 104 with respect to the mask 200 on acquired ultrasound image data 400. For example, the reticle positioning module 150 may receive a current ultrasound probe 104 position and orientation from the position sensing system 112 and/or may access the position data that was associated with the acquired ultrasound image data by the position sensing system 112. The reticle positioning module 150 may be configured to dynamically update the position and orientation of the reticle 300 overlaid onto the ultrasound image data 400 in substantially real-time as the ultrasound probe 104 is moved to provide real-time positioning feedback that an ultrasound operator may use to move the probe 104 to align the reticle 300 with the mask 200. The alignment of the reticle 300 with the mask 200 corresponds with the ultrasound probe 104 being located at the appropriate position and orientation to acquire the desired pre-defined view of the anatomical structure.

The reticle positioning module 150 may generate a reticle 300 having primary reticle element 302, at least one lateral reticle element 304, at least one elevational reticle element 306, and a reticle rotational indicator 308. Each of the reticle elements 302, 304, 306 may be a shape, such as a circle, oval, square, rectangle, star, or any suitable shape. The reticle elements 302, 304, 306 may be the same size or smaller than the target areas 202, 204, 206 of the mask 200. The at least one lateral reticle element 304 may be located on a first side, a second side, or both sides of the primary reticle element 302. The at least one elevational reticle element 306 may be located above the primary reticle element 302, below the primary reticle element 302, or both above and below the primary reticle element 302. In a representative embodiment, the number of lateral and elevational reticle elements 304, 306 may correspond with the number of lateral and elevational target areas 204, 206 of the mask 200. The reticle rotational indicator 308 may extend at an angle from the primary reticle element 302 to between a lateral reticle element 304 and an elevational reticle element 306. In various embodiments, the reticle rotational indicator 308 is not centered (i.e., 45 degrees) between the lateral reticle element 304 and the elevational reticle element 306 so that the alignment of the reticle 300 with the mask 200 is possible in only one ultrasound probe 104 orientation.

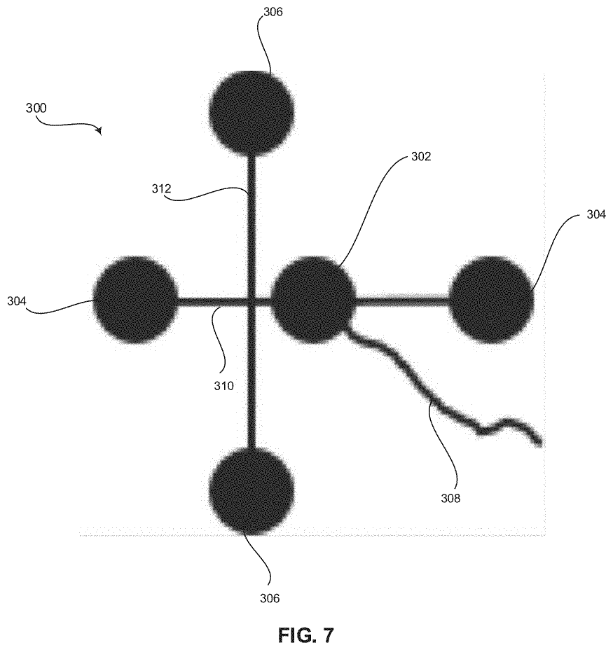

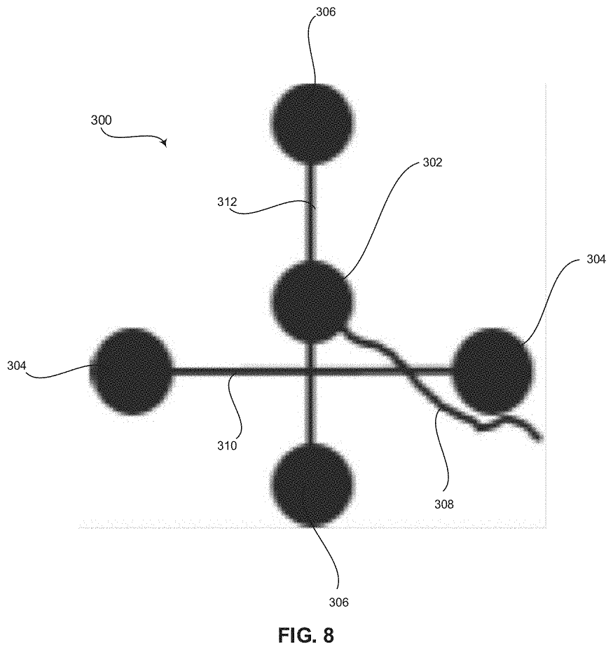

In the exemplary embodiments illustrated in FIGS. 2-8 and 10, the reticle 300 includes a centrally-located primary reticle element 302 with a lateral reticle element 304 on both sides of the primary reticle element 302 and an elevational reticle element 306 both above and below the primary reticle element 302. In certain embodiments having a lateral reticle element 304 on both sides of the primary reticle element 302, the lateral reticle elements 304 may be connected by a lateral connecting element 310. In various embodiments having an elevational reticle element 306 both above and below the primary reticle element 302, the elevational reticle elements 306 may be connected by an elevational connecting element 312. The lateral and elevational connecting elements 310, 312 along with the primary reticle element 302 may provide enhanced visualization of the tilt of a corresponding ultrasound probe 104. For example, as illustrated in FIGS. 7 and 8, the primary reticle element 302 may be shown above, below, to a left side, or to a right side of a point where the lateral and elevational connecting elements 310, 312 intersect. The position of the primary reticle element 302 with respect to the intersecting point of the lateral and elevational connecting elements 310, 312 may provide visual feedback with respect to the amount and direction of current tilt of an ultrasound probe 104. As an example, the position of the primary reticle element 302 with respect to the intersecting point of the lateral and elevational connecting elements 310, 312 in FIG. 7 indicates that the ultrasound probe 104 is currently tilted laterally to the right. As another example, the position of the primary reticle element 302 with respect to the intersecting point of the lateral and elevational connecting elements 310, 312 in FIG. 8 indicates that the ultrasound probe 104 is currently tilted in a forward elevational direction.

In the exemplary embodiments illustrated in FIGS. 2-8 and 10, the reticle 300 includes a reticle rotational indicator 308 that corresponds with a rotational orientation of the associated ultrasound probe 104. The alignment of the reticle elements 302, 304, 306 and the position of the reticle rotational indicator 308 provides visual feedback related to a current orientation (e.g., rotation and tilt) of an ultrasound probe 104 so that the ultrasound probe 104 may be manipulated by an operator to match the orientation of the target areas 202, 204, 206 and the mask rotational indicator 208 of the mask 200. The position and orientation of the reticle 300 with respect to a position and orientation of the mask 200 provides visual feedback for moving the ultrasound probe 104 to match the target areas 202, 240, 206 of the mask 200. As described in more detail below, manipulating an ultrasound probe 104 to align the reticle elements 302, 304, 306, 308 of the reticle 300 with the target areas 202, 204, 206 and rotational indicator 208 of the mask 200 results in the ultrasound probe 104 being positioned and oriented to obtain the desired pre-defined view of the anatomical structure. The reticle 300 may be overlaid with the mask 200 onto ultrasound image data 400 presented at the display system 134 as shown in FIG. 10 and described in more detail below. Additionally and/or alternatively, the reticle 300 and mask 200 may be presented at other portions of an ultrasound display at the display system 134, such as in a side, top, or bottom panel of the display.

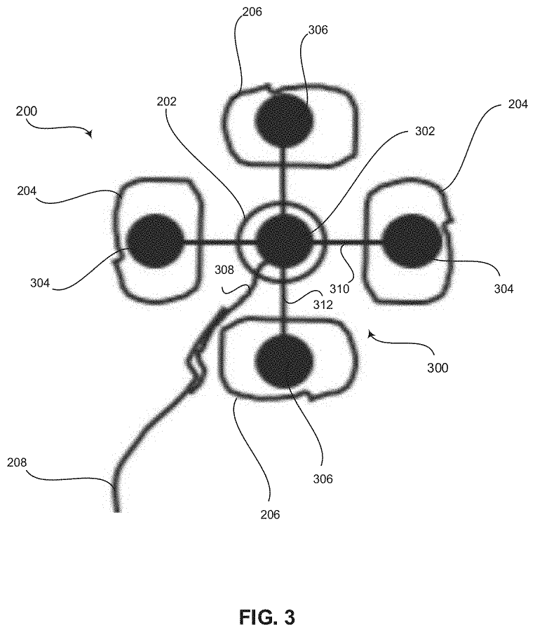

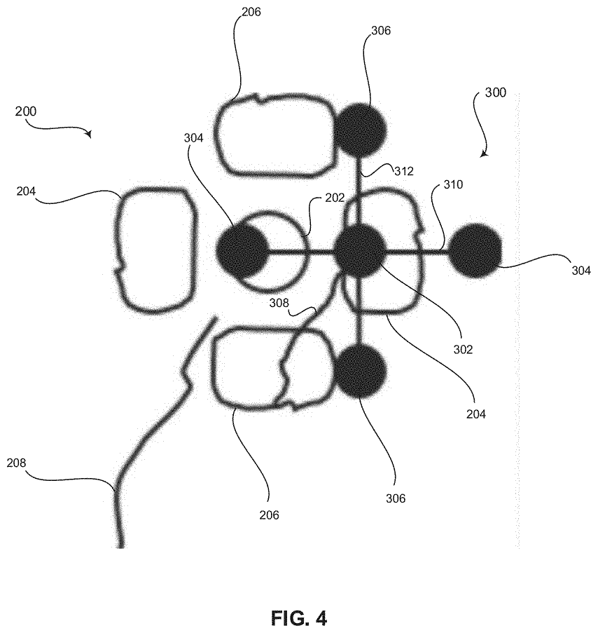

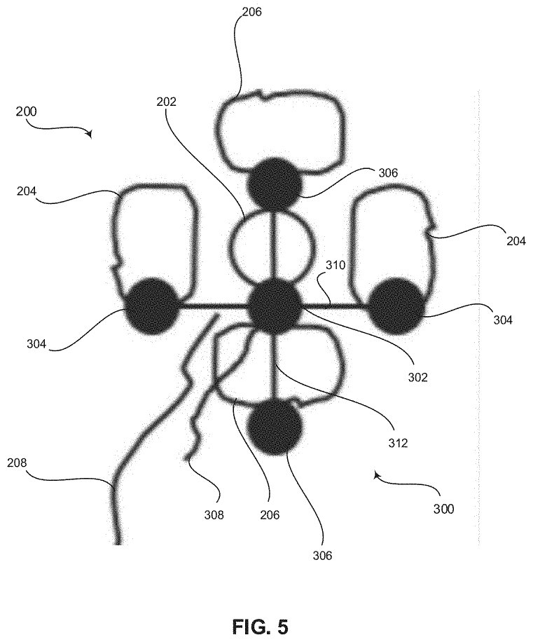

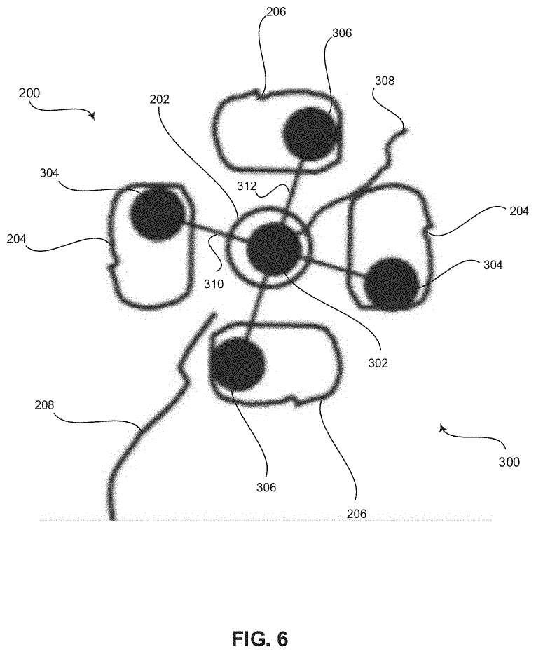

FIG. 2 illustrates an exemplary mask 200 and reticle 300 configured to provide enhanced visualization of ultrasound probe 104 positioning feedback, in accordance with exemplary embodiments. FIG. 3 illustrates an exemplary reticle 300 aligned with an exemplary mask 200 that corresponds with a correctly positioned ultrasound probe 104, in accordance with various embodiments. FIG. 4 illustrates an exemplary reticle 300 laterally misaligned with an exemplary mask 200 to provide feedback for moving an ultrasound probe 104 to a correct position and orientation, in accordance with exemplary embodiments. FIG. 5 illustrates an exemplary reticle 300 misaligned in an elevation direction with an exemplary mask 200 to provide feedback for moving an ultrasound probe 104 to a correct position and orientation, in accordance with various embodiments. FIG. 6 illustrates an exemplary reticle 300 rotationally misaligned with an exemplary mask 200 to provide feedback for moving an ultrasound probe 104 to a correct position and orientation, in accordance with exemplary embodiments.

Referring to FIGS. 2-6, the mask 200 comprises a primary target area 202, lateral target areas 204 on both sides of the primary target area 202, elevational target areas 206 above and below the primary target area 202, and a mask rotational indicator 208. Each of the target areas 202, 204, 206 may be an enclosed shape, such as a circle, oval, square, rectangle, or any suitable shape. The mask rotational indicator 208 may extend at an angle from the primary target area 202. For example, the mask rotational indictor 208 may extend between a lateral target area 204 and an elevational target area 206. In various embodiments, the rotational indicator 208 is not centered (i.e., 45 degrees) between the lateral target area 204 and the elevational target area 206 so that the alignment of the reticle 300 with the mask 200 is possible in only one ultrasound probe 104 orientation. In various embodiments, the mask may have a non-military and non-technical appearance. For example, the mask 200 may have an appearance similar to a plant or flower, such as a four leaf clover or the like, where the lateral target areas 204 and elevational target areas 206 are similar to leaves or pedals and the mask rotational indicator 208 is similar to a stem. The alignment of the target areas 202, 204, 206 and the position of a mask rotational indicator 208 corresponds with a target orientation (e.g., rotation and tilt) of an ultrasound probe 104 for obtaining the pre-defined view of the anatomical structure. The position of the mask 200 with respect to a position of the reticle 300 associated with the current position of the ultrasound probe 104 corresponds with a target position of the ultrasound probe 104 for obtaining the pre-defined view of the anatomical structure.

The reticle 300 comprises a primary reticle element 302, a lateral reticle element 304 on both sides of the primary reticle element 302, an elevational reticle element 306 both above and below the primary reticle element 302, and a reticle rotational indicator 308. The lateral reticle elements 304 may be connected by a lateral connecting element 310. The elevational reticle elements 306 may be connected by an elevational connecting element 312. The reticle rotational indicator 308 corresponds with a rotational orientation of the associated ultrasound probe 104. The reticle rotational indicator 308 may extend at an angle from the primary reticle element 302. For example, the reticle rotational indictor 308 may extend between a lateral reticle element 304 and an elevational reticle element 306. In various embodiments, the reticle rotational indicator 308 is not centered (i.e., 45 degrees) between the lateral reticle element 304 and the elevational reticle element 306 so that the alignment of the reticle 300 with the mask 200 is possible in only one ultrasound probe 104 orientation.

Referring to FIG. 3, the reticle 300 is shown in alignment with the mask 200. For example, the primary reticle element 302 is positioned and oriented within the enclosed primary target area 202 of the mask 200. Each of the lateral reticle elements 304 are positioned within respective enclosed lateral target areas 204 of the mask 200. Each of the elevational reticle elements 306 are positioned within respective enclosed elevational target areas 206 of the mask 200. The reticle rotational indicator 308 extends in a same direction and may overlap with the mask rotational indicator 208.

Referring to FIG. 4, the reticle 300 is shown laterally misaligned with the mask 200. For example, the primary reticle element 302, lateral reticle elements 304, elevational reticle elements 306, and reticle rotational indicator 308 are positioned laterally to the right side of the corresponding primary target area 202, lateral target areas 204, elevational target areas 206, and mask rotational indicator 208 of the mask 200. The position of the reticle 200 with respect to the mask 300 provides visual feedback directing an ultrasound operator to move the ultrasound probe 104 to the left to align the reticle 300 with the mask 200.

Referring to FIG. 5, the reticle 300 is shown misaligned in an elevation direction with the mask 200. For example, the primary reticle element 302, lateral reticle elements 304, elevational reticle elements 306, and reticle rotational indicator 308 are positioned below the corresponding primary target area 202, lateral target areas 204, elevational target areas 206, and mask rotational indicator 208 of the mask 200. The position and orientation of the reticle 200 with respect to the mask 300 provides visual feedback directing an ultrasound operator to move the ultrasound probe 104 forward in the elevational direction to align the reticle 300 with the mask 200.

Referring to FIG. 6, the reticle 300 is shown rotationally misaligned with the mask 200. For example, the primary reticle element 302, lateral reticle elements 304, elevational reticle elements 306, and reticle rotational indicator 308 are oriented approximately one hundred and eighty (180) degrees from the corresponding primary target area 202, lateral target areas 204, elevational target areas 206, and mask rotational indicator 208 of the mask 200. The reticle rotation indicator 308, for example, extends in an opposite direction from the mask rotational indicator 208. The orientation of the reticle 200 with respect to the mask 300 provides visual feedback directing an ultrasound operator to rotate the ultrasound probe 104 approximately 180 degrees to align the reticle 300 with the mask 200.

FIG. 7 illustrates an exemplary reticle 300 having a lateral tilt, in accordance with various embodiments. FIG. 8 illustrates an exemplary reticle 300 having an elevational tilt, in accordance with exemplary embodiments. Referring to FIGS. 7 and 8, a reticle 300 comprises a primary reticle element 302, a lateral reticle element 304 on both sides of the primary reticle element 302, an elevational reticle element 306 both above and below the primary reticle element 302, and a reticle rotational indicator 308. The lateral reticle elements 304 are connected by a lateral connecting element 310. The elevational reticle elements 306 are connected by an elevational connecting element 312. The lateral and elevational connecting elements 310, 312 intersect. The location of the intersection between the lateral and elevational connecting elements 310, 312 with respect to the primary reticle element 302 provides feedback as to the tilt of an associated ultrasound probe. For example, the primary reticle element 302 may be shown above, below, to a left side, to a right side, or at the point where the lateral and elevational connecting elements 310, 312 intersect. The ultrasound probe 104 that is not tilted may have the primary reticle element 302 positioned at the point where the lateral and elevational connecting elements 310, 312 intersect as shown in FIGS. 2-6 and 10. The ultrasound probe 104 is tilted laterally to the right if the primary reticle element 302 is positioned on the lateral connecting element 310 to the right of the intersecting point as illustrated in FIG. 7. The ultrasound probe 104 is tilted laterally to the left if the primary reticle element 302 is positioned on the lateral connecting element 310 to the left of the intersecting point. The ultrasound probe 104 is tilted forward in an elevational direction if the primary reticle element 302 is positioned on the elevational connecting element 312 above the intersecting point as illustrated in FIG. 8. The ultrasound probe 104 is tilted rearward in an elevational direction if the primary reticle element 302 is positioned on the elevational connecting element 312 below the intersecting point. Accordingly, the position of the primary reticle element 302 with respect to the intersecting point of the lateral and elevational connecting elements 310, 312 may provide visual feedback with respect to the amount and direction of current tilt of an ultrasound probe 104.

FIG. 10 illustrates an exemplary mask 200 and reticle 300 overlaid on an ultrasound image 400 to provide enhanced visualization of ultrasound probe 104 positioning feedback, in accordance with exemplary embodiments. Referring to FIG. 10, a mask 200, a reticle 300, and an image label 402 are superimposed on an ultrasound image 400. The ultrasound image 400 having the overlaid mask 200, reticle 300, and label 400 may be presented at the display system 134. The mask 200 includes a primary target area 202, lateral target areas 204, elevational target areas 206, and a mask rotational indicator 208. The mask 200 corresponds with a target ultrasound probe 104 position and orientation for acquiring ultrasound image data 400 of a pre-defined view of an identified anatomical structure. The reticle 300 comprises a primary reticle element 302, lateral reticle elements 304, elevational reticle elements 306, and a reticle rotational indicator 308. The lateral reticle elements 304 are connected by a lateral connecting element 310. The elevational reticle elements 306 are connected by an elevational connecting element 312. The reticle 300 corresponds with a current ultrasound probe 104 position and orientation. The image label 402 corresponds with the pre-defined view of an anatomical structure associated with the mask 200. For example, the pre-defined view may correspond with a biparietal diameter (BPD) measurement as shown in FIG. 10. The reticle 300 illustrated in FIG. 10 appears aligned in orientation (e.g., tilt and rotation) but is misaligned in position (e.g., lateral and elevational). For example, an ultrasound operator may move the ultrasound probe 104 forward and to the left to align the reticle 300 with the mask 200. The position and orientation of the reticle 300 dynamically updates in substantially real-time on the ultrasound image 400 at the display system 134 as the ultrasound probe is moved, rotated, and/or tilted. The ultrasound image data 400 is dynamically presented at the display system 134 as it is acquired by the ultrasound probe 104. The ultrasound data 400 presented at the display system 134 is the pre-defined view of the anatomical structure when the reticle 300 is aligned with the mask 200.

Referring again to FIG. 1, the signal processor 132 may include an imaging system action module 160 that comprises suitable logic, circuitry, interfaces and/or code that may be operable to execute an imaging system action in response to the alignment of the reticle 300 with the mask 200. For example, the imaging system action module 160 may be configured to automatically store the acquired ultrasound image data 400 when the reticle 300 is aligned with the mask 200. The acquired ultrasound image data 400 may be stored in archive 138 or any suitable data storage medium. As another example, the imaging system action module 160 may be configured to automatically provide measurement tools when the reticle 300 is matched to the mask 200. The measurement tools may include caliper tools, structure outlining tools, or any suitable measurement tools. For example, the caliper tools may be executed to receive start and end points selections of a caliper measurement via the user input module 130. The structure outlining tools may be executed to receive a user directive via the user input module 130 to outline selected anatomical structure in the ultrasound image data 400 for performing an area measurement or any suitable measurement. In various embodiments, the imaging system action module 160 may be configured to automatically perform one or more measurements corresponding to the pre-defined view of the anatomical structure. For example, the imaging system action module 160 may automatically perform a biparietal diameter (BPD) or head circumference (HC) measurement if the pre-defined view is of the fetal head. As another example, the imaging system action module 160 may automatically perform an abdominal circumference (AC) measurement if the pre-defined view is of the fetal abdomen. The imaging system action module 160 may automatically perform a femur diaphysis length (FDL) measurement if the pre-defined view is of the fetal femur. The measurements performed automatically or via the measurement tools may be stored by the imaging system action module 160 in archive 138 or any suitable data storage medium.

Still referring to FIG. 1, the teaching engine 170 may comprise suitable logic, circuitry, interfaces and/or code that may be operable to train the neurons of the deep neural network(s) of the mask positioning module 140 to automatically identify anatomical structures. For example, the teaching engine 170 may train the deep neural networks of the mask positioning module 140 using databases(s) of classified images. As an example, a mask positioning module 140 deep neural network may be trained by the teaching engine 170 with images of a particular anatomical structure to train the mask positioning module 140 with respect to the characteristics of the particular anatomical structure, such as the appearance of structure edges, the appearance of structure shapes based on the edges, the positions of the shapes relative to landmarks in the ultrasound image data 400, and the like. In certain embodiments, the anatomical structure may be a fetus and the structural information may include information regarding the edges, shapes, and positions of a fetal head, abdomen, femur, and/or the like. In various embodiments, the databases of training images may be stored in the archive 138 or any suitable data storage medium. In certain embodiments, the training engine 170 and/or training image databases may be external system(s) communicatively coupled via a wired or wireless connection to the ultrasound system 100.

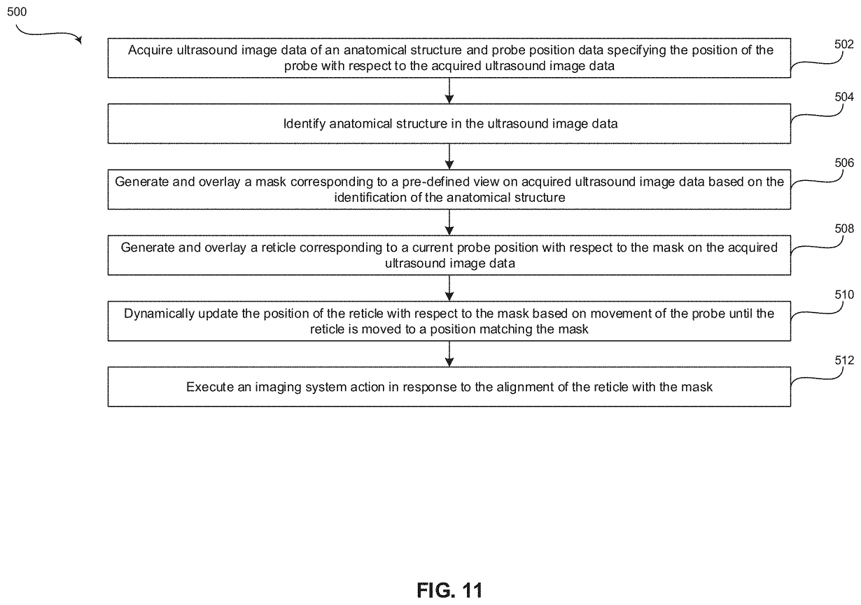

FIG. 11 is a flow chart 500 illustrating exemplary steps 502-512 that may be utilized for providing enhanced visualization of ultrasound probe 104 positioning feedback, in accordance with various embodiments. Referring to FIG. 11, there is shown a flow chart 500 comprising exemplary steps 502 through 512. Certain embodiments may omit one or more of the steps, and/or perform the steps in a different order than the order listed, and/or combine certain of the steps discussed below. For example, some steps may not be performed in certain embodiments. As a further example, certain steps may be performed in a different temporal order, including simultaneously, than listed below.

At step 502, an ultrasound system 100 may acquire ultrasound image data 400 of an anatomical structure and probe position data specifying the position and orientation of the ultrasound probe 104 with respect to the acquired ultrasound image data 400. For example, the ultrasound system 100 may acquire ultrasound image data 400 with an ultrasound probe 104 having a position sensing system 112. The ultrasound probe 104 may provide ultrasound image data corresponding with an anatomical structure, such as a fetus or any suitable anatomical structure. The position sensing system 112 may provide probe position data that is provided to a signal processor 132 of the ultrasound system 100. The signal processor 132 may associate the probe position data with the corresponding ultrasound image data 400 acquired at each of the ultrasound probe 104 positions and orientations.

At step 504, a signal processor 132 of the ultrasound system 100 may identify and/or receive an identification of anatomical structure in the ultrasound image data 400. For example, a mask positioning module 140 of the signal processor 132 may receive the identification via a user input module 130 during acquisition of the ultrasound image data by an ultrasound operator. As another example, the mask positioning module 140 of the signal processor 132 may employ image detection and/or machine learning algorithms to identify anatomical structure in the ultrasound image data 400. In various embodiments, the image detection and/or machine learning algorithms of the mask positioning module 140 may include deep neural network(s) made up of an input layer, output layer, and one or more hidden layers between the input and output layer. Each of the layers may perform a processing function before passing the processed ultrasound information to a subsequent layer for further processing. The processing performed by the mask positioning module 140 deep neural network may identify anatomical structure in ultrasound image data 400. The anatomical structure may be an organ such as a liver, heart, or the like. The anatomical structure may be a fetus and can include fetal structures such as a fetal head, fetal abdomen, fetal femur, and/or any suitable structure of a fetus.

At step 506, the signal processor 132 may generate and overlay a mask 200 corresponding to a pre-defined view on acquired ultrasound image data 400 based on the identification of the anatomical structure at step 504. For example, various anatomical structures may be associated with a pre-defined view providing a desired view of each of the anatomical structures. The information regarding the pre-defined views of each anatomical structure may be stored in archive 138 or any suitable data storage medium. The mask positioning module 140 of the signal processor 132 may access the information related to the pre-defined view of the identified anatomical structure and may generate and superimpose a mask 200 on the ultrasound image data 400 to provide a target position and orientation of an ultrasound probe 104. The mask 200 may include a primary target area 202, at least one lateral target area 204, at least one elevational target area 206, and a mask rotational indicator 208. Each of the target areas 202, 204, 206 may be an enclosed shape, such as a circle, oval, square, rectangle, or any suitable shape. The size of the target areas 202, 204, 206 may correspond with an amount of alignment precision for obtaining the pre-defined view of the anatomical structure. The at least one lateral target area 204 may be located on a first side, a second side, or both sides of the primary target area 202. The at least one elevational target area 206 may be located above the primary target area 202, below the primary target area 202, or both above and below the primary target area 202. The mask rotational indicator 208 may extend at an angle from the primary target area 202 between a lateral target area 204 and an elevational target area 206. The alignment of the target areas 202, 204, 206 and a position of a mask rotational indicator 208 may correspond with a target rotation and tilt of an ultrasound probe 104 for obtaining the pre-defined view of the anatomical structure. The position and orientation of the mask 200 may correspond with a target position and orientation of the ultrasound probe 104 for obtaining the pre-defined view of the anatomical structure.

At step 508, the signal processor 132 may generate and overlay a reticle 300 corresponding to a current probe 104 position and orientation with respect to the mask 200 on the acquired ultrasound image data 400. For example, the reticle positioning module 150 may generate a reticle 300 having a primary reticle element 302, at least one lateral reticle element 304, at least one elevational reticle element 306, and a reticle rotational indicator 308. Each of the reticle elements 302, 304, 306 may be a shape, such as a circle, oval, square, rectangle, star, or any suitable shape. The reticle elements 302, 304, 306 may be the same size or smaller than the target areas 202, 204, 206 of the mask 200. The at least one lateral reticle element 304 may be located on a first side, a second side, or both sides of the primary reticle element 302. The at least one elevational reticle element 306 may be located above the primary reticle element 302, below the primary reticle element 302, or both above and below the primary reticle element 302. The number of lateral and elevational reticle elements 304, 306 corresponds with the number of lateral and elevational target areas 204, 206 of the mask 200. The reticle rotational indicator 308 may extend at an angle from the primary reticle element 302 to between a lateral reticle element 304 and an elevational reticle element 306. The reticle positioning module 150 of the signal processor 132 may receive a current ultrasound probe 104 position and orientation from the position sensing system 112 and/or may access the position and orientation data that was associated with the acquired ultrasound image data by the position sensing system 112. The reticle positioning module 150 superimposes the generated reticle 300 on the ultrasound image data 400 relative the mask 200 based on the position and orientation data.

At step 510, the signal processor 132 may dynamically update the position and orientation of the reticle 300 with respect to the mask 200 based on movement of the probe 104 until the reticle 300 is moved to a position and orientation matching the mask 200. For example, the reticle positioning module 150 of the signal processor 132 may dynamically update the position and orientation of the reticle 300 overlaid onto the ultrasound image data 400 in substantially real-time as the ultrasound probe 104 is moved, rotated, and/or titled to provide real-time positioning feedback that an ultrasound operator may use to move the probe 104 to align the reticle 300 with the mask 200. The alignment of the reticle 300 with the mask 200 corresponds with the ultrasound probe 104 being located at the appropriate position and orientation to acquire the desired pre-defined view of the anatomical structure.

At step 512, the signal processor 132 may execute an imaging system action in response to the alignment of the reticle 300 with the mask 200. For example, an imaging system action module 160 of the signal processor 132 may be configured to automatically store the acquired ultrasound image data 400 of the pre-defined view, automatically provide measurement tools for performing a measurement of the acquired ultrasound image data 400 of the pre-defined view, and/or automatically perform a measurement of the acquired ultrasound image data 400 of the pre-defined view. The ultrasound image data and/or measurements may be stored by the imaging system action module 160 in archive 138 or any suitable data storage medium.

Aspects of the present disclosure provide a method 500 and system 100 for providing enhanced visualization of ultrasound probe 104 positioning feedback. In accordance with various embodiments, the method 500 may comprise receiving 502, by at least one processor 132, 140, 150, 160, ultrasound image data 400 and probe position data corresponding with the ultrasound image data 400. The method 500 may comprise presenting 506 at a display system 134, by the at least one processor 132, 140, a mask 200 defining a target position and orientation of an ultrasound probe 104 that corresponds to a pre-defined ultrasound image view of anatomical structure. The mask 200 may comprise a primary target area 202, at least one lateral target area 204 positioned laterally from the primary target area 202, and at least one elevational target area 206 positioned in an elevational direction from the primary target area 202. The method 500 may comprise presenting 508, 510 at the display system 134, by the at least one processor 132, 150, a reticle 300 having a reticle position and orientation corresponding to a position and orientation of the ultrasound probe 104 based on the probe position data. The reticle position and orientation presented at the display system 134 is dynamically updated with respect to the mask 200 based on the probe position data and in response to movement of the ultrasound probe 104. The reticle 300 may comprise a primary reticle element 302 configured to align with the primary target area 202 of the mask 200 when the ultrasound probe 104 is located at the target position and orientation. The reticle 300 may comprise at least one lateral reticle element 304 positioned laterally from the primary reticle element 302 and configured to align with the at least one lateral target area 204 of the mask 200 when the ultrasound probe 104 is located at the target position and orientation. The reticle 300 may comprise at least one elevational reticle element 306 positioned in an elevational direction from the primary reticle element 302 and configured to align with the at least one elevational target area 206 of the mask 200 when the ultrasound probe 104 is located at the target position and orientation. The method 500 may comprise executing 512, by the at least one processor 132, 160, an imaging system action based on the reticle 300 aligning with the mask 200 in response to movement of the ultrasound probe 104 to the target position and orientation for acquiring the ultrasound image data 400 of the pre-defined ultrasound image view of the anatomical structure.

In a representative embodiment, the method 500 may comprise identifying 504 the anatomical structure in the ultrasound image data 400. The pre-defined ultrasound image view of the anatomical structure may be based on the anatomical structure identified in the ultrasound image data 400. In an exemplary embodiment, the anatomical structure may be automatically identified by the processor 132, 140 based on machine-learning algorithms. In various embodiments, the mask 200 and the reticle 300 are superimposed on the ultrasound image data 400. In certain embodiments, the ultrasound image data 400 and probe position data corresponding with the ultrasound image data 400 may be acquired by the ultrasound probe 104 having a position sensing system 112. In a representative embodiment, the mask 200 may comprise a mask rotational indicator 208 extending at an angle from the primary target area 202 between one of the at least one lateral target area 204 and one of the at least one elevational target area 206. The reticle 300 may comprise a reticle rotational indicator 308 extending at an angle from the primary reticle element 302 between one of the at least one lateral reticle element 304 and one of the at least one elevational reticle element 206. The reticle rotational indicator 308 may be configured to align with the mask rotational indicator 208 when the ultrasound probe 104 is located at the target position and orientation.

In an exemplary embodiment, the at least one lateral target area 204 may be one lateral target area 204 on each lateral side of the primary target area 202. The at least one elevational target area 206 may be one elevational target area 206 in each elevational direction of the primary target area 202. The at least one lateral reticle element 304 may be one lateral reticle element 304 on each lateral side of the primary reticle element 302. The at least one elevational reticle element 306 may be one elevational reticle element 306 in each elevational direction of the primary reticle element 302. In certain embodiments, the imaging system action may be automatically storing the ultrasound image data 400 of the pre-defined ultrasound image view of the anatomical structure. The imaging system action may be automatically providing measurement tools for performing a measurement within the ultrasound image data 400 of the pre-defined ultrasound image view of the anatomical structure. The imaging system action may be automatically performing a measurement within the ultrasound image data 400 of the pre-defined ultrasound image view of the anatomical structure.

Various embodiments provide a system 100 for providing enhanced visualization of ultrasound probe 104 positioning feedback. The system 100 may comprise an ultrasound probe 104, a display system 134, and at least one processor 132, 140, 150, 160. The at least one processor 132, 140, 150, 160 may be configured to receive ultrasound image data 400 and probe position data corresponding with the ultrasound image data 400. The at least one processor 132, 140 may be configured to present, at the display system 134, a mask 200 defining a target position and orientation of the ultrasound probe 104 that corresponds to a pre-defined ultrasound image view of anatomical structure. The mask 200 may comprise a primary target area 202, at least one lateral target area 204 positioned laterally from the primary target area 202, and at least one elevational target area 206 positioned in an elevational direction from the primary target area 202. The at least one processor 132, 150 may be configured to present, at the display system 134, a reticle 300 having a reticle position and orientation corresponding to a position and orientation of the ultrasound probe 104 based on the probe position data. The reticle position and orientation presented at the display system 134 may be dynamically updated with respect to the mask 200 based on the probe position data and in response to movement of the ultrasound probe 104. The reticle 300 may comprise a primary reticle element 302 configured to align with the primary target area 202 of the mask 200 when the ultrasound probe 104 is located at the target position and orientation. The reticle 300 may comprise at least one lateral reticle element 304 positioned laterally from the primary reticle element 302 and configured to align with the at least one lateral target area 204 of the mask 200 when the ultrasound probe 104 is located at the target position and orientation. The reticle 300 may comprise at least one elevational reticle element 306 positioned in an elevational direction from the primary reticle element 302 and configured to align with the at least one elevational target area 206 of the mask 200 when the ultrasound probe 104 is located at the target position and orientation. The at least one processor 132, 160 may be configured to execute an imaging system action based on the reticle 300 aligning with the mask 200 in response to movement of the ultrasound probe 104 to the target position and orientation for acquiring the ultrasound image data 400 of the pre-defined ultrasound image view of the anatomical structure.

In certain embodiments, the at least one processor 132, 140 may be configured to automatically identify the anatomical structure in the ultrasound image data 400 based on machine-learning algorithms. The pre-defined ultrasound image view of the anatomical structure may be based on the anatomical structure automatically identified in the ultrasound image data 400. In various embodiments, the ultrasound probe 104 may comprise a position sensing system 112 configured to provide the probe position data. In a representative embodiment, the mask 200 and the reticle 300 may be superimposed on the ultrasound image data 400. In an exemplary embodiment, the mask 200 may comprise a mask rotational indicator 208 extending at an angle from the primary target area 202 between one of the at least one lateral target area 204 and one of the at least one elevational target area 206. The reticle may comprise a reticle rotational indicator 308 extending at an angle from the primary reticle element 302 between one of the at least one lateral reticle element 304 and one of the at least one elevational reticle element 306. The reticle rotational indicator 308 may be configured to align with the mask rotational indicator 208 when the ultrasound probe 104 is located at the target position and orientation.

In various embodiments, the at least one lateral target area 204 may be one lateral target area 204 on each lateral side of the primary target area 202. The at least one elevational target area 206 may be one elevational target area 206 in each elevational direction of the primary target area 202. The at least one lateral reticle element 304 may be one lateral reticle element 304 on each lateral side of the primary reticle element 302. The at least one elevational reticle element 306 may be one elevational reticle element 306 in each elevational direction of the primary reticle element 302. In a representative embodiment, the imaging system action may be automatically storing the ultrasound image data 400 of the pre-defined ultrasound image view of the anatomical structure. The imaging system action may be automatically providing measurement tools for performing a measurement within the ultrasound image data 400 of the pre-defined ultrasound image view of the anatomical structure. The imaging system action may be automatically performing a measurement within the ultrasound image data 400 of the pre-defined ultrasound image view of the anatomical structure.

Certain embodiments provide a non-transitory computer readable medium having stored thereon, a computer program having at least one code section. The at least one code section is executable by a machine for causing the machine to perform steps 500. The steps 500 may include receiving 502 ultrasound image data 400 and probe position data corresponding with the ultrasound image data 400. The steps 500 may comprise displaying 506 a mask 200 defining a target position and orientation of an ultrasound probe 104 that corresponds to a pre-defined ultrasound image view of anatomical structure. The mask may comprise a primary target area 202, at least one lateral target area 204 positioned laterally from the primary target area 202, and at least one elevational target area 206 positioned in an elevational direction from the primary target area 202. The steps 500 may comprise displaying 508, 510 a reticle 300 having a reticle position and orientation corresponding to a position and orientation of the ultrasound probe 104 based on the probe position data. The reticle position and orientation may be dynamically updated with respect to the mask 200 based on the probe position data and in response to movement of the ultrasound probe 104. The reticle 200 may comprise a primary reticle element 302 configured to align with the primary target area 202 of the mask 200 when the ultrasound probe 104 is located at the target position and orientation. The reticle 200 may comprise at least one lateral reticle element 304 positioned laterally from the primary reticle element 302 and configured to align with the at least one lateral target area 204 of the mask 200 when the ultrasound probe 104 is located at the target position and orientation. The reticle 200 may comprise at least one elevational reticle element 306 positioned in an elevational direction from the primary reticle element 302 and configured to align with the at least one elevational target area 206 of the mask 200 when the ultrasound probe 104 is located at the target position and orientation. The steps 500 may comprise executing 512 an imaging system action based on the reticle 300 aligning with the mask 200 in response to movement of the ultrasound probe 104 to the target position and orientation for acquiring the ultrasound image data 400 of the pre-defined ultrasound image view of the anatomical structure.

In an exemplary embodiment, the mask 200 and the reticle 300 are superimposed on the ultrasound image data 400. In various embodiments, the mask 200 may comprise a mask rotational indicator 208 extending at an angle from the primary target area 202 between one of the at least one lateral target area 204 and one of the at least one elevational target area 206. The reticle 300 may comprise a reticle rotational indicator 308 extending at an angle from the primary reticle element 302 between one of the at least one lateral reticle element 304 and one of the at least one elevational reticle element 306. The reticle rotational indicator 308 may be configured to align with the mask rotational indicator 208 when the ultrasound probe 104 is located at the target position and orientation.

In a representative embodiment, the at least one lateral target area 204 may be one lateral target area 204 on each lateral side of the primary target area 202. The at least one elevational target area 206 may be one elevational target area 206 in each elevational direction of the primary target area 202. The at least one lateral reticle element 304 may be one lateral reticle element 304 on each lateral side of the primary reticle element 302. The at least one elevational reticle element 306 may be one elevational reticle element 306 in each elevational direction of the primary reticle element 302. In certain embodiments, the imaging system action may be automatically storing the ultrasound image data 400 of the pre-defined ultrasound image view of the anatomical structure. The imaging system action may be automatically providing measurement tools for performing a measurement within the ultrasound image data 400 of the pre-defined ultrasound image view of the anatomical structure. The imaging system action may be automatically performing a measurement within the ultrasound image data 400 of the pre-defined ultrasound image view of the anatomical structure.

As utilized herein the term “circuitry” refers to physical electronic components (i.e. hardware) and any software and/or firmware (“code”) which may configure the hardware, be executed by the hardware, and or otherwise be associated with the hardware. As used herein, for example, a particular processor and memory may comprise a first “circuit” when executing a first one or more lines of code and may comprise a second “circuit” when executing a second one or more lines of code. As utilized herein, “and/or” means any one or more of the items in the list joined by “and/or”. As an example, “x and/or y” means any element of the three-element set {(x), (y), (x, y)}. As another example, “x, y, and/or z” means any element of the seven-element set {(x), (y), (z), (x, y), (x, z), (y, z), (x, y, z)}. As utilized herein, the term “exemplary” means serving as a non-limiting example, instance, or illustration. As utilized herein, the terms “e.g.,” and “for example” set off lists of one or more non-limiting examples, instances, or illustrations. As utilized herein, circuitry is “operable” and/or “configured” to perform a function whenever the circuitry comprises the necessary hardware and code (if any is necessary) to perform the function, regardless of whether performance of the function is disabled, or not enabled, by some user-configurable setting.

Other embodiments may provide a computer readable device and/or a non-transitory computer readable medium, and/or a machine readable device and/or a non-transitory machine readable medium, having stored thereon, a machine code and/or a computer program having at least one code section executable by a machine and/or a computer, thereby causing the machine and/or computer to perform the steps as described herein providing enhanced visualization of ultrasound probe positioning feedback.

Accordingly, the present disclosure may be realized in hardware, software, or a combination of hardware and software. The present disclosure may be realized in a centralized fashion in at least one computer system, or in a distributed fashion where different elements are spread across several interconnected computer systems. Any kind of computer system or other apparatus adapted for carrying out the methods described herein is suited.

Various embodiments may also be embedded in a computer program product, which comprises all the features enabling the implementation of the methods described herein, and which when loaded in a computer system is able to carry out these methods. Computer program in the present context means any expression, in any language, code or notation, of a set of instructions intended to cause a system having an information processing capability to perform a particular function either directly or after either or both of the following: a) conversion to another language, code or notation; b) reproduction in a different material form.

While the present disclosure has been described with reference to certain embodiments, it will be understood by those skilled in the art that various changes may be made and equivalents may be substituted without departing from the scope of the present disclosure. In addition, many modifications may be made to adapt a particular situation or material to the teachings of the present disclosure without departing from its scope. Therefore, it is intended that the present disclosure not be limited to the particular embodiment disclosed, but that the present disclosure will include all embodiments falling within the scope of the appended claims.

What is claimed is:

1. A method comprising:
receiving, by at least one processor, ultrasound image data and probe position data corresponding with the ultrasound image data;
presenting at a display system, by the at least one processor, a mask defining a target position and orientation of an ultrasound probe that corresponds to a pre-defined ultrasound image view of anatomical structure, the mask comprising:
a primary target area,
at least one lateral target area positioned laterally from the primary target area, and
at least one elevational target area positioned in an elevational direction from the primary target area;
presenting at the display system, by the at least one processor, a reticle having a reticle position and orientation corresponding to a position and orientation of the ultrasound probe based on the probe position data, wherein the reticle position and orientation presented at the display system is dynamically updated with respect to the mask based on the probe position data and in response to movement of the ultrasound probe, the reticle comprising:
a primary reticle element configured to align with the primary target area of the mask when the ultrasound probe is located at the target position and orientation,
at least one lateral reticle element positioned laterally from the primary reticle element and configured to align with the at least one lateral target area of the mask when the ultrasound probe is located at the target position and orientation, and
at least one elevational reticle element positioned in an elevational direction from the primary reticle element and configured to align with the at least one elevational target area of the mask when the ultrasound probe is located at the target position and orientation; and
executing, by the at least one processor, an imaging system action based on the reticle aligning with the mask in response to movement of the ultrasound probe to the target position and orientation for acquiring the ultrasound image data of the pre-defined ultrasound image view of the anatomical structure.

2. The method of claim 1, comprising identifying the anatomical structure in the ultrasound image data, wherein the pre-defined ultrasound image view of the anatomical structure is based on the anatomical structure identified in the ultrasound image data.

3. The method of claim 2, wherein the anatomical structure is automatically identified by the processor based on machine-learning algorithms.

4. The method of claim 1, wherein the mask and the reticle are superimposed on the ultrasound image data.

5. The method of claim 1, wherein the ultrasound image data and probe position data corresponding with the ultrasound image data is acquired by the ultrasound probe having a position sensing system.

6. The method of claim 1, wherein:
the mask comprises a mask rotational indicator extending at an angle from the primary target area between one of the at least one lateral target area and one of the at least one elevational target area, and
the reticle comprises a reticle rotational indicator extending at an angle from the primary reticle element between one of the at least one lateral reticle element and one of the at least one elevational reticle element, the reticle rotational indicator configured to align with the mask rotational indicator when the ultrasound probe is located at the target position and orientation.

7. The method of claim 1, wherein:
the at least one lateral target area is one lateral target area on each lateral side of the primary target area,
the at least one elevational target area is one elevational target area in each elevational direction of the primary target area,
the at least one lateral reticle element is one lateral reticle element on each lateral side of the primary reticle element, and
the at least one elevational reticle element is one elevational reticle element in each elevational direction of the primary reticle element.

8. The method of claim 1, wherein the imaging system action is one or more of:
automatically storing the ultrasound image data of the pre-defined ultrasound image view of the anatomical structure,
automatically providing measurement tools for performing a measurement within the ultrasound image data of the pre-defined ultrasound image view of the anatomical structure, and
automatically performing a measurement within the ultrasound image data of the pre-defined ultrasound image view of the anatomical structure.

9. A system comprising:
an ultrasound probe;
a display system; and
at least one processor configured to:
receive ultrasound image data and probe position data corresponding with the ultrasound image data;
present, at the display system, a mask defining a target position and orientation of the ultrasound probe that corresponds to a pre-defined ultrasound image view of anatomical structure, the mask comprising:
a primary target area,
at least one lateral target area positioned laterally from the primary target area, and
at least one elevational target area positioned in an elevational direction from the primary target area;
present, at the display system, a reticle having a reticle position and orientation corresponding to a position and orientation of the ultrasound probe based on the probe position data, wherein the reticle position and orientation presented at the display system is dynamically updated with respect to the mask based on the probe position data and in response to movement of the ultrasound probe, the reticle comprising:
a primary reticle element configured to align with the primary target area of the mask when the ultrasound probe is located at the target position and orientation,
at least one lateral reticle element positioned laterally from the primary reticle element and configured to align with the at least one lateral target area of the mask when the ultrasound probe is located at the target position and orientation, and
at least one elevational reticle element positioned in an elevational direction from the primary reticle element and configured to align with the at least one elevational target area of the mask when the ultrasound probe is located at the target position and orientation; and
execute an imaging system action based on the reticle aligning with the mask in response to movement of the ultrasound probe to the target position and orientation for acquiring the ultrasound image data of the pre-defined ultrasound image view of the anatomical structure.

10. The system of claim 9, wherein the at least one processor is configured to automatically identify the anatomical structure in the ultrasound image data based on machine-learning algorithms, and wherein the pre-defined ultrasound image view of the anatomical structure is based on the anatomical structure automatically identified in the ultrasound image data.

11. The system of claim 9, wherein the ultrasound probe comprises a position sensing system configured to provide the probe position data.

12. The system of claim 9, wherein the mask and the reticle are superimposed on the ultrasound image data.

13. The system of claim 9, wherein:
the mask comprises a mask rotational indicator extending at an angle from the primary target area between one of the at least one lateral target area and one of the at least one elevational target area, and
the reticle comprises a reticle rotational indicator extending at an angle from the primary reticle element between one of the at least one lateral reticle element and one of the at least one elevational reticle element, the reticle rotational indicator configured to align with the mask rotational indicator when the ultrasound probe is located at the target position and orientation.

14. The system of claim 9, wherein:
the at least one lateral target area is one lateral target area on each lateral side of the primary target area,
the at least one elevational target area is one elevational target area in each elevational direction of the primary target area,
the at least one lateral reticle element is one lateral reticle element on each lateral side of the primary reticle element, and
the at least one elevational reticle element is one elevational reticle element in each elevational direction of the primary reticle element.

15. The system of claim 9, wherein the imaging system action is one or more of:
automatically storing the ultrasound image data of the pre-defined ultrasound image view of the anatomical structure,
automatically providing measurement tools for performing a measurement within the ultrasound image data of the pre-defined ultrasound image view of the anatomical structure, and
automatically performing a measurement within the ultrasound image data of the pre-defined ultrasound image view of the anatomical structure.

16. A non-transitory computer readable medium having stored thereon, a computer program having at least one code section, the at least one code section being executable by a machine for causing the machine to perform steps comprising:
receiving ultrasound image data and probe position data corresponding with the ultrasound image data;
displaying a mask defining a target position and orientation of an ultrasound probe that corresponds to a pre-defined ultrasound image view of anatomical structure, the mask comprising:
a primary target area,
at least one lateral target area positioned laterally from the primary target area, and
at least one elevational target area positioned in an elevational direction from the primary target area;
displaying a reticle having a reticle position and orientation corresponding to a position and orientation of the ultrasound probe based on the probe position data, wherein the reticle position and orientation is dynamically updated with respect to the mask based on the probe position data and in response to movement of the ultrasound probe, the reticle comprising:
a primary reticle element configured to align with the primary target area of the mask when the ultrasound probe is located at the target position and orientation,
at least one lateral reticle element positioned laterally from the primary reticle element and configured to align with the at least one lateral target area of the mask when the ultrasound probe is located at the target position and orientation, and
at least one elevational reticle element positioned in an elevational direction from the primary reticle element and configured to align with the at least one elevational target area of the mask when the ultrasound probe is located at the target position and orientation; and
executing an imaging system action based on the reticle aligning with the mask in response to movement of the ultrasound probe to the target position and orientation for acquiring the ultrasound image data of the pre-defined ultrasound image view of the anatomical structure.

17. The non-transitory computer readable medium of claim 16, wherein the mask and the reticle are superimposed on the ultrasound image data.

18. The non-transitory computer readable medium of claim 16, wherein:
the mask comprises a mask rotational indicator extending at an angle from the primary target area between one of the at least one lateral target area and one of the at least one elevational target area, and
the reticle comprises a reticle rotational indicator extending at an angle from the primary reticle element between one of the at least one lateral reticle element and one of the at least one elevational reticle element, the reticle rotational indicator configured to align with the mask rotational indicator when the ultrasound probe is located at the target position and orientation.

19. The non-transitory computer readable medium of claim 16, wherein:
the at least one lateral target area is one lateral target area on each lateral side of the primary target area,
the at least one elevational target area is one elevational target area in each elevational direction of the primary target area,
the at least one lateral reticle element is one lateral reticle element on each lateral side of the primary reticle element, and
the at least one elevational reticle element is one elevational reticle element in each elevational direction of the primary reticle element.

20. The non-transitory computer readable medium of claim 16, wherein the imaging system action is one or more of:
automatically storing the ultrasound image data of the pre-defined ultrasound image view of the anatomical structure,
automatically providing measurement tools for performing a measurement within the ultrasound image data of the pre-defined ultrasound image view of the anatomical structure, and
automatically performing a measurement within the ultrasound image data of the pre-defined ultrasound image view of the anatomical structure.
